(19) United States
(12) Patent Application Publication
(10) Pub. No.: US 2024/0016413 A1
(43) Pub. Date: Jan. 18, 2024

(54) SYSTEMS AND METHODS FOR COUGH DETECTION

(71) Applicant: KONINKLIJKE PHILIPS N.V., EINDHOVEN (NL)

(72) Inventors: David Aaron Troxell, Monroeville, PA (US); Samer Bou Jawde, Cambridge, MA (US); Seunghyun Lee, Monroeville, PA (US); Peter Douglas Hill, Pittsburgh, PA (US)

(21) Appl. No.: 18/195,404

(22) Filed: May 10, 2023

Related U.S. Application Data
(60) Provisional application No. 63/389,028, filed on Jul. 14, 2022.

(51) Int. Cl. A61B 5/08; G16H 40/67; G16H 50/20

(52) U.S. Cl. CPC A61B 5/0823; G16H 40/67; G16H 50/20; A61B 2562/0219

(57) ABSTRACT

An embodiment provides techniques for distinguishing between breathing events based on sensor data obtained from one or more wearable sensors. In one example, sensor data is obtained that includes one or more of a sensor signal and descriptive metadata of the sensor signal. Processing is applied to distinguish between a cough and another breathing event based on the sensor data, and an indication of a cough is provided.

### CROSS-REFERENCE TO RELATED APPLICATIONS

This patent application claims the priority benefit under 35 U.S.C. § 119(e) of U.S. Provisional Application No. 63/389,028, filed on Jul. 14, 2022, the contents of which are herein incorporated by reference.

### BACKGROUND OF THE INVENTION

### 1. Field of the Invention

The disclosed subject matter generally pertains to remote patient monitoring with respect to respiratory conditions. Certain disclosed subject matter relates to technologies for accurately distinguishing between breathing events such as coughs and sneezes as represented in sensor data.

### 2. Description of the Related Art

Chronic obstructive pulmonary disease (COPD) is a progressive, life-threatening lung disease that causes breathlessness and predisposes the sufferer to exacerbations and serious illness. COPD is associated with progressive, irreversible worsening of airflow limitation secondary to alveolar wall destruction, bronchiolar narrowing, and airway inflammation. The primary cause of the disease is exposure to tobacco smoke (including second-hand smoke), with other risk factors including indoor and outdoor air pollution, as well as occupational dusts and fumes. According to the World Health Organization (WHO), COPD is the third leading cause of death worldwide, claiming 3.23 million deaths in 2019 alone. WHO predicts an increase in COPD due to an increase in smoking prevalence, as well as aging populations in many countries.

The average patient with COPD experiences two acute exacerbation-COPD (AE-COPD) events annually accounting for a significant consumption of health care resources. AE-COPD has been described as a clinical diagnosis that is made when a patient with COPD fits one or more of the following criteria: sustained (e.g., 24-28 hr) increase in cough, sputum production, and/or dyspnea. AE-COPD is associated with a wide range of clinical consequences including progressive respiratory failure.

Cough and sputum production are reported in between 60-80% of patients with COPD, and chronic cough and mucus hypersecretion are associated with faster lung function decline, increased exacerbation rate and increased mortality in COPD. Cough is known to be blunted during sleep, though the exact reasons are not fully understood. Nocturnal coughing can be an indication of sleep fragmentation—also important in the evaluation and monitoring of COPD, as both sleep and cough are vital functions. In a large longitudinal sample, the predictive value of respiratory symptoms (including cough and sputum production) for hospitalization was examined over a 12-year period and cough had the greatest predictive value for subsequent hospital admission due to respiratory disease and due to COPD. Cough is therefore considered an important biomarker of changes in respiratory baseline status for COPD patients.

### SUMMARY OF THE INVENTION

Conventionally, detection of coughing and other breathing events has been performed using sensors that are adapted to provide an indication of audible coughing or movement of the body associated with coughing. However, the resolution of such sensors is not adequate to reliably distinguish between breathing events, such as coughing and sneezing. Further, often such sensors are not tailored to patient comfort and therefore long-term wear and monitoring is inhibited.

Accordingly, an embodiment provides technologies that permit a wearable sensor to provide sensor data of sufficient accuracy to reliably distinguish between breathing events of interest. Further, embodiments provide for use of composite or multiple sensor signals obtained from more than one sensor. Also, embodiments allow for long term monitoring via wearable sensor, making accurate prediction and detection of breathing events possible in a highly sensitive manner.

In summary, an embodiment includes a method, comprising: obtaining, using a set of one or more processors, sensor data comprising one or more of a sensor signal received from a force sensor worn by a patient and descriptive metadata of the sensor signal; distinguishing, using the set of one or more processors, between a cough and another breathing event based on the sensor data; and providing, using the set of one or more processors, an indication of a cough.

In an embodiment, coughs are distinguished from other breathing events including one or more of a sneeze, throat clearing, a sigh, and tidal breathing. The distinguishing may include utilizing one or more features of the sensor data to identify a cough characteristic associated with inspiration, for example the cough characteristic may include a signal morphology that occurs after inspiration. By way of specific example, the cough characteristic may include one or more of: a pair of signal peaks occurring within a predetermined time period; and a ratio of slopes relating one of the pair of signal peaks prior to a trough and another of the pair of signal peaks following the trough. In an example embodiment, the predetermined time period may be less than about 1.0 seconds. In an example embodiment, the ratio may be about 1.5 or more. In an example embodiment, the cough characteristic comprises a standard deviation of slopes relating signal peaks to respective troughs. In an embodiment, the cough characteristic comprises a predetermined pattern of signal peak intensities.

An embodiment may obtain the sensor data from a force sensing capacitor. An embodiment may obtain the sensor data from two or more sensors and use signal that combines data of the two or more sensors to distinguish between breathing events. The two or more sensors may include one or more of: (a) a resistive, capacitive, inductive, or fiber-optic strain sensor; (b) an impedance sensor; (c) a heart rate sensor; and (d) one or more movement sensors comprising an accelerometer, a gyroscope, a magnetometer, or an inertial measurement unit (IMU).

In an embodiment, distinguishing between breathing events may include identifying one or more features in training sensor data, providing the training sensor data to a model based on the one or more features, and using the model after training to classify the sensor data as a cough or another breathing event.

As will become apparent from reviewing this specification, methods, devices, systems, and products are provided for implementing the various embodiments.

The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of the invention.

### BRIEF DESCRIPTION OF THE DRAWINGS

FIG. 1 illustrates an example system.

FIG. 1A illustrates example signals that may be used in combination.

FIG. 2A-2D illustrate examples of signal characteristics for different breathing events.

FIG. 3 illustrates an example method.

FIG. 3A illustrates an example of monitoring cough frequency and/or intensity over time for prediction and detection.

FIG. 3B illustrates an example of monitoring a combination of signals over time for prediction and detection.

FIG. 4 illustrates an example method.

FIG. 5 illustrates a diagram of example system components.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, e.g., through one or more intermediate parts or components, so long as a link occurs. As used herein, “operatively coupled” means that two or more elements are coupled so as to operate together or are in communication, unidirectional or bidirectional, with one another. As used herein, the term “number” shall mean one or an integer greater than one (i.e., a plurality). As used herein a “set” shall mean one or more.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

Although it is known that respiratory symptoms have predictive value, e.g., occurrence of acute exacerbations of COPD (AE-COPD), hospitalization, etc., it is difficult to accurately track the respiratory symptoms in a useful manner. For example, AE-COPD events are often linked to bacterial or viral infections and a sneeze may be the first symptom of an infection. However, a sneeze has similar characteristics to a cough: a large insufflation, vocal cord closure, thoracic compression followed by an explosive volume of air when the vocal cords open. While both sneezing and coughing are important biomarkers for AE-COPD risk stratification in individuals with COPD, distinguishing between these (and other breathing events such as throat clearing, a sigh, vocalization or phonation, tidal breathing, etc.) is important because different biomarkers may carry different predictive value and weight. For example, cough detection can be trended to provide insight as to whether the daily cough rate is climbing, potentially signalling a deterioration of a disease state or decreasing indicating recovery from illness or respiratory stabilization.

Conventionally, cough and sneeze detection can be accomplished at a coarse level using a variety of methods such as use of microphones for audible recordings, accelerometers, flow sensors, pressure transducers, photoplethysmography sensors (PPG), etc. An embodiment introduces a sensing technology allowing for granular analysis of breathing events, such as cough and sneeze detection. An embodiment utilizes a miniaturized force sensing capacitor (FSC) sensor to provide granular sensor data related to a breathing event that may be used in isolation to distinguish between various breathing event types. An embodiment may also include using multiple sensing technologies to consider a combination of signals for distinguishing between breathing events such as a by producing a cough or sneeze signals that are more reliable than the signal obtained from a single sensor alone. Combination-signal based cough or sneeze detection improves accuracy of the signal detection by using the signal from multiple, unique sensors to confirm the signal detection. A combination of signals is likely to facilitate accurate signal detection even when an artifact is present in one or more signals, as other sensors may be free of artifact.

The description now turns to the figures. The illustrated example embodiments will be best understood by reference to the figures. The following description is intended only by way of example, and simply illustrates certain example embodiments.

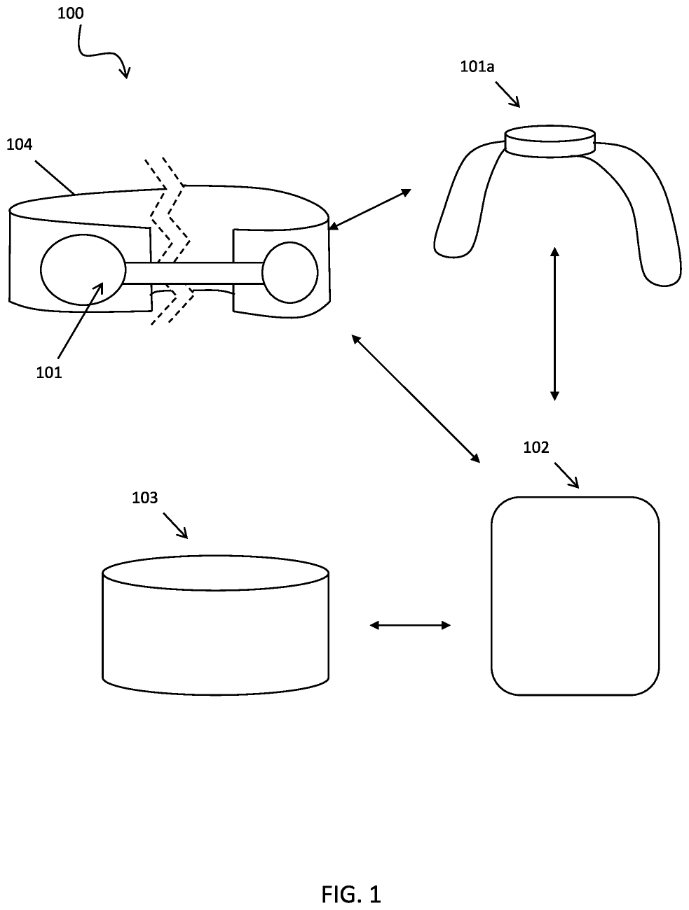

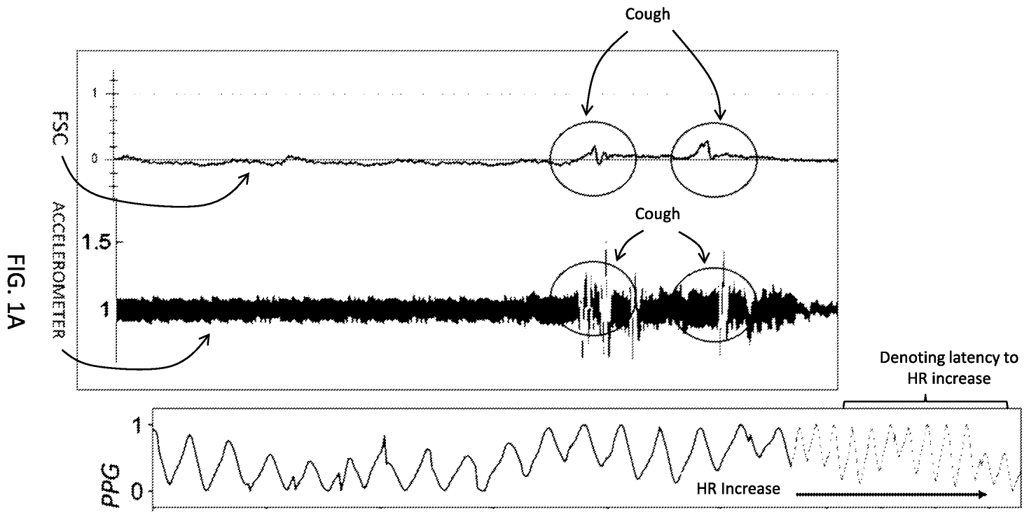

FIG. 1 illustrates an example system 100 that may be used to perform accurate detection of a breathing event such as a cough. An embodiment provides a wearable sensor 101, for example incorporated into a band 104 for use during remote monitoring of a patient. In the illustrated example of FIG. 1, system 100 includes a wearable sensor 101 in the form of a force sensor, such as a force sensing capacitor (FSC). FSC 101 is a lightweight and low-cost force sensor, yielding highly reproducible results and is very sensitive, making it an ideal sensor for providing a wearable that is discreet and comfortable, for example when integrated into band 104 with elastic properties that surrounds the waistline, thorax, or neck. FSC 101 may be used for accurately detecting breathing events such as cough and sneeze reflexes. One example embodiment may be implemented using an FCS 101 with a range set to 4.5 Newtons (N), such as a SINGLETACT sensor as produced by PPS UK Limited, Glasgow, United Kingdom, e.g., a SINGLETACT force-sensitive capacitor that uses molded silicon between two layers of polyimide to construct a 0.35 mm thick sensor having a nominal capacitance of 75 pF, increasing by 2.2 pF when the rated force is applied. Force sensing technology contemplated herein includes but is not necessarily limited to strain gauges, piezoelectric, inductive, resistive, and capacitive sensors.

In an embodiment FSC 101 is an ultra-low power sensor and circuit that detects breathing events including coughing and sneezing events. As further described herein, upon detecting a precursor to a cough or a cough, additional sensor(s) included in a device (collectively indicated at 101a) may be energized and data from them analyzed to confirm the presence of a cough, type of cough, cough frequency, cough force or intensity, as well as differentiation from other breathing events, such as a sneeze reflex. Further, the unique ability to energize additional sensors 101a based on biometric thresholds, e.g., as detected from FSC 101, may facilitate the use of other higher energy consuming sensors such as a pulse oximetry (e.g., using either a reflectance or transmission probe) to expand the clinical utility of the biosensor as well as provide additional signatures to characterize the cough or sneeze signal from artifact.

As further described herein, rules-based or machine learning processes (or other pattern or feature recognition techniques) may be employed to classify the signals or sensor data, e.g., from FSC 101, including identifying data indicating when to activate signals from the additional sensor(s) 101a. An embodiment may utilize an appropriate sensor technology as a primary sensor to sense chest movement induced by cough, including but not limited to resistive, capacitive, inductive, and fiber-optic strain sensors, impedance sensors such as transthoracic impedance sensors, and movement sensors such as an accelerometer, a gyroscope, a magnetometer, or an inertial measurement unit (IMU).

In one embodiment, system 100 is used to implement a method for discreet cough and sneeze reflex detection by use of FSC 101 housed in or attached to a means 104 for ease of application to the patient being monitored. For example, FSC 101 may be included with or attached to a strap, belt, patch, elastic band, or like element or housing that firmly holds FSC 101 against the skin or clothing of the patient to sense compressive, strain, tensile, lateral, or horizontal forces due to breathing events. Thoracic and/or abdominal compression that occurs during the compressive phase of a cough reflex provides the force necessary for FSC 101 activation and the distinctive cough signal to be produced and analyzed. FSC 101 is operatively coupled with a software program and a classifier for signal analysis and cough and sneeze detection.

Optionally, one or more layers of foam, elastic or shock-absorbing materials such as rubber or silicone, or other materials such as fabrics or plastics may be applied to one or more surfaces of FSC 101 so as to partially insulate the surface or surfaces of FSC 101 from the forces being sensed at the skin or clothing of the patient during use. As one having ordinary skill in the art will appreciate, factors such as the mounting location of the FCS 101 on the body of the patient, the force with which the FSC 101 is pressed against the skin or clothing of the patient, and the properties of any intervening layers or materials, can affect the signal quality, and should be optimized to minimize signal artifacts.

With respect to sensor data processing, this may take place in a variety of system components or combination thereof. For example, system 100 may include a device 102 that is operatively coupled to FSC 101 or another sensor device 101a. For example, device 102 may be a user device such as a smart phone, tablet computer or similar. FSC 101 and device 102 are operatively coupled and device 102 may obtain sensor data from FSC 101, for example raw waveform data, which may be digitized and communicated from a radio or other communication module of FSC 101 to device 102. In one example, a low-power wireless communication mechanism between FSC 101 and device 102 is provided. Device 102 may in turn store and process sensor data locally or may communicate with a remote device 103, for example a server or database processing and storing electronic health information.

In one example, device 102 may be used to detect a cough using sensor data from FSC 101 and activate additional sensor(s) 101a. An embodiment therefore uses FSC 101 to perform intelligent power management for additional sensor(s) 101a that are associated with larger power consumption. FSC 101 is a type of ultra-low power consuming device and therefore use of FSC-based cough detection holds benefits in that high power consuming sensors such as a photoplethysmogram (PPG) sensor or a pulse oximeter can remain in an idle/stand-by state reducing power consumption until FSC 101 detects a cough or other pertinent biometric threshold and subsequently energizes the higher power consuming sensor.

Thus, additional sensor(s) 101a may be included in or communicate with various components of system 100. For example, a device such as a smart watch with additional sensor(s) 101a such as a PPG sensor may provide data to an operatively coupled device such as device 102 or may act in concert with FSC 101 or other sensors to collect or process biomarker data, e.g., add cough data to a health application resident on a device including additional sensors 101a and/or device 102. A device including additional sensor(s) 101a may provide raw or processed data to device 102, e.g., PPG data, PPG data processed into a heart rate, etc. In some embodiment where multiple sensor devices are utilized (whether housed in the same device or separate devices), a correlation may be made between the sensor data, for example synchronizing the sensor data readings in time, as explained in the example of FIG. 1A.

FIG. 1A illustrates a series of sensor data measurements for FSC 101 as well as additional sensors 101a, in this case an accelerometer and a PPG sensor. As illustrated, a breathing event (here a series of coughs) represented in the sensor data of FSC 101 may be correlated in time with accelerometer data and heart rate (HR) data, with the HR data being offset in time due to latency of the physiological response of the heart to the breathing event.

An embodiment therefore combines cough detection from FSC 101 with additional sensing technologies creating a combination cough detection technique. In the example of FIG. 1A, a combination of signals is created by considering signals from FSC 101 together with additional sensors 101a, e.g., an accelerometer as well as a PPG sensor. Each sensor has a unique signature which will improve detection accuracy while eliminating noise originating from sensor artifact which may lead to erroneous cough or sneeze classification. For example, cough has been observed to produce a chronotropic effect. HR typically increases by up to 30% within four beats after the last cough reflex. This characteristic provides a distinct signature or rule that facilitates accurate cough detection. Using the PPG sensor to extract both the respiratory waveform morphology as well as to set a HR threshold confirms the accuracy of the cough detection utilizing two distinct signals from a single additional sensor.

While multiple sensor data types are illustrated in FIG. 1A, an embodiment may utilize a plurality of the same sensors, e.g., placed at different locations. For example, several FSCs 101 could be incorporated at several locations, such as at different and distant locations. Each of the FSCs 101 could be continuously on (as they are low power) or one or more of them could be on and trigger the others, e.g., others would be turned on as a cough is being detected for the purpose of power management (as explained herein). In this case, it might be possible to add the signals of the several differently located sensors to form a composite signal, e.g., to increase the signal to noise ratio. Such a technique could further help to estimate the intensity of the cough more accurately from the peak of the composite signal. This, in addition to the number of coughs within an episode and their time occurrence, would shed light on the status of the disease progression as well as a patient's response to therapy. In an embodiment FSC 101 may be coupled with or replaced with one of a plurality of sensing technologies such as resistive capacitance sensors, strain gauges, microphones, etc.

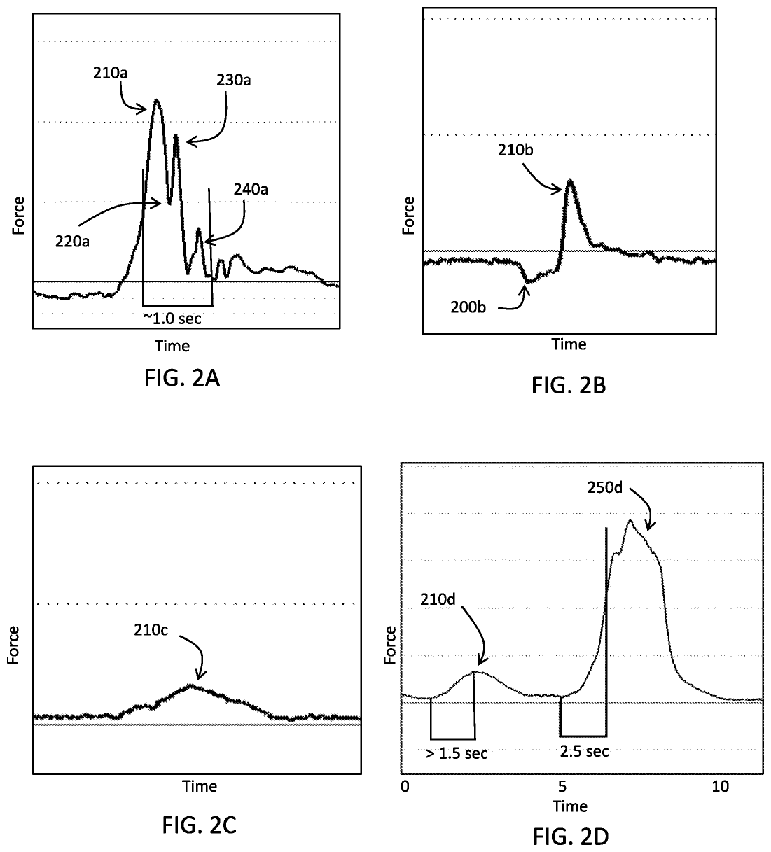

An embodiment receives sensor data, for example from FSC 101 and/or another sensor device, and analyzes it to distinguish between various breathing events with increased accuracy as compared to the state of the art. By way of example, FIG. 2(A-D) illustrate waveforms obtained from FSC 101 for various breathing events that exhibit characteristics or features that may be used to distinguish between breathing event types. For example, a cough reflex has three main phases: (1) a deep inspiration creating a large lung volume; (2) glottis closure and simultaneous intercostal and abdominal contraction (a decrease in alveolar volume is associated with a large increase in intra-alveolar pressure due to the inverse relationship between volume and pressure); and 3) glottis opening, followed by high expiratory flow rates due to the large pressure gradient between the alveoli and atmospheric pressure. In contrast, a sneeze is associated with irritation of the mucous membranes of the nose or throat producing a deep inspiration followed by depression of the soft palate and palatine uvula with elevation of the back of the tongue that partially closes the passage to the mouth. In a sneeze, air bursts suddenly through the lungs with variable force, expelling mucus containing foreign particles or irritants from the oral and nasal cavities. In the second and third phases of the cough reflex, which include intercostal and abdominal compression and characteristic trough and subsequent spike, FSC 101 may be activated and create a unique signature or cough characteristic as further described herein.

As may be appreciated from review of FIG. 2A, which depicts a series of coughs, FSC 101 provides sensor data that may be analyzed to distinguish the cough based on wave morphology directly or indirectly via use of descriptive metadata characterizing the wave morphology, e.g., numerically. For example, descriptive metadata may include but is not necessarily limited to sensor data indicating a force or pressure value, a change in force or pressure value, a maximum force or pressure value, a minimum force or pressure value, an instantaneous rate of change of the force or pressure value, or an average rate of change of the force or pressure value as well as processed force or pressure data, for example force or pressure values converted or transformed into descriptive values such as timing data, intensity data, frequency data, slope data, ratios of slopes, slope standard deviations, etc., as further described herein.

As shown in FIG. 2A, a large inspiration and subsequent glottic closure creates a pressure peak 210a. Compressive force associated with abdominal and thoracic cage compression subsequently creates a reduction of the thoracic cage circumference, resulting in a pressure decay identified by trough 220a on the cough waveform example of FIG. 2A. Thereafter, an explosive phase is identifiable as a cough characteristic that includes pressure reversal and subsequent pressure spike 230a. As shown in the example of FIG. 2A, two individual cough reflexes on the cough waveform are visible with peaks at 230a and 240a. It should also be noted that the cough reflex expels a large amount of lung volume so that subsequent cough reflexes in a peal of coughs, e.g., peak 240a, will demonstrate a substantially linear decline in peak cough flow (PCF) rates as compared to earlier peaks, e.g., peak 230a. Using FSC 101 for cough detection reveals a similar pattern where each subsequent cough reflex is associated with a smaller pressure spike, as in the example of FIG. 2A.

As shown in FIG. 2B, sensor data of FSC 101 for a sneeze reflex reveals a unique signature distinguishing the pressure waveform from a cough. Here, there is a brief, sharp inhalation followed by abdominal & thoracic compression creating a transient pressure drop 200b, followed by a sharp pressure spike 210b with the explosive sneeze reflex thereafter, resulting in rapid waveform decay.

In FIG. 2C is illustrated sensor data from FSC 101 for a throat-clearing event, where a sound made at the back of the throat by tightly constricting the laryngopharyngeal tissues and vibrating the palatoglossal arch and the vocal folds while exhaling. This may be done with the mouth slightly opened or completely closed. In the example of FIG. 2C, the resultant waveform is that of a small peak 210c, somewhat similar to a mound that results from tidal breathing (210d of FIG. 2D). Each of these breathing events, i.e., throat-clearing, and tidal breathing, have relatively symmetric waveforms characterized by their regularity and substantial smoothness. That is, each signal is characterized by a small, rounded pressure waveform during the vibratory exhalation phase of throat clearing or during normal tidal breathing. It is noted that FSC 101 produces substantially a baseline response for phonation or vocalization.

Referring to FIG. 2D, it may be appreciated that features of the waveform produced by FSC 101 may be used to distinguish between breathing events. In one example, an embodiment may use timing information to distinguish between breathing events. In the example of FIG. 2D, the time take for tidal breathing (e.g., less than 1.5 seconds) is substantially less than the time taken for a sigh or yawn (e.g., about 2.5 seconds in this example). Further, the waveform produced by FSC 101 may be used to distinguish between breathing events based on amplitude, as indicated by the large force difference between tidal breathing peak 210d at 2.6N and a peak 250d at 3.8N associated with a sigh. In some embodiments, a combination of timing data and amplitude may be utilized, e.g., to identify an intensity characteristic or feature of the waveform.

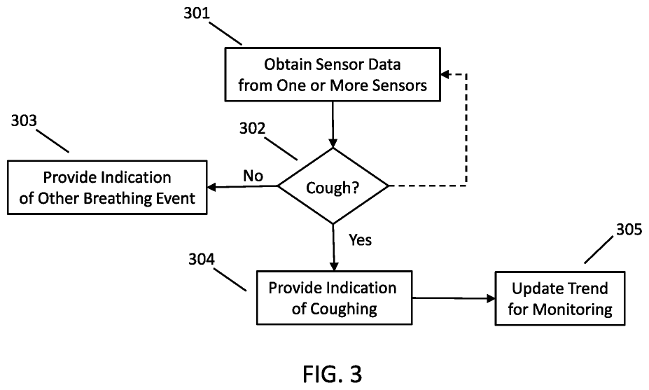

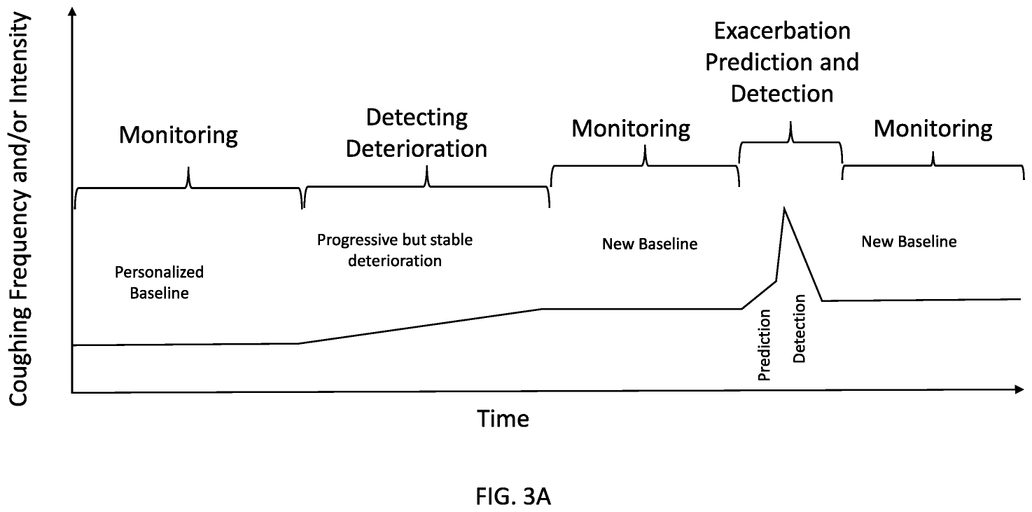

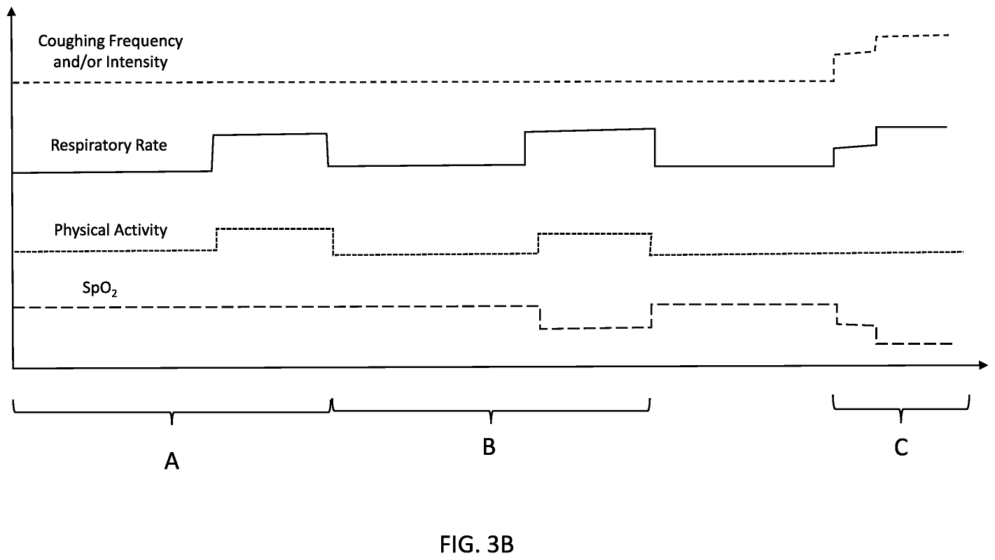

As illustrated in FIG. 3, an embodiment may utilize sensor data, for example from FSC 101 waveform, a PPG, other sensors, or a combination thereof, to identify features that are characteristic of the breathing events. For example, an embodiment may be coded with a rules-based system that identifies characteristics or features of breathing events, such as cough characteristics including a series of peaks separated by a trough within a predetermined time, etc., and uses such descriptive metadata to classify a given set of sensor data as inclusive of a cough, another breathing event, or no event. Similarly, an embodiment may utilize a machine learning process to train a neural network to differentiate data sensor signals based on breathing events of interest. In such an embodiment, labeled training data, similar to the information represented in FIG. 2A-D may be supplied to a neural network, which extracts features useful in classifying the waveforms into different categories. Once trained, a model may be deployed to a device such as device 102 to evaluate the sensor data as it is collected.

As shown in FIG. 3, an embodiment obtains sensor data at 301, e.g., from FSC 101 and/or other sensors. An analysis is performed on the sensor data at 302, e.g., to determine if a cough is detected in the sensor data. If so, an indication is provided at 304. Otherwise, an alternative indication (e.g., of a sneeze, etc.) may be provided. Similarly, if no breathing event is detected, the process may return to a data obtaining or listening model.

As further illustrated in FIG. 3, monitoring, deterioration detection, and exacerbation prediction using a cough monitoring sensor may be performed by an embodiment. For example, an embodiment may provide ongoing monitoring of a breathing condition, for example updating a trend of coughing data at 305 responsive to detecting coughing at 304. Disease severity can be captured by the both the frequency of coughing as well as the intensity of coughing. Thus, by monitoring both frequency and intensity, the condition or severity of the disease could be monitored. Additionally, exacerbations are often preceded by increase of signals indicative of increasing frequency and intensity of coughing. Hence, monitoring could also be used to trigger an exacerbation prediction. A baseline for each patient may be set to provide personal assessment and care, as reported for example on a dashboard or user interface.

For example, FIG. 3A provides a trend of coughing frequency and/or intensity plotted against time. In an embodiment, the trend of frequency and intensity data for a breathing event of interest, e.g., coughing, may be used to categorize the patient's disease progression and provide predictions and detections of specific events, e.g., AE-COPD events. In the example of FIG. 3A, coughing frequency and/or intensity data, e.g., obtained from FSC 101 and/or other sensor(s), is plotted over time and compared with trends, e.g., trending upwards at a given rate indicates deterioration, and/or thresholds, e.g., a trend upwards in frequency and/or intensity over a given threshold yields a prediction of an event and/or a detection of an event. Such information may be reported in a dashboard such as a web page or user interface, similar to that illustrated in FIG. 3A.

Referring to FIG. 3B, in a case where additional sensors are utilized, such as pulse oximeter and physical activity sensors in combination with an FSC, their data is also obtained, e.g., at 301 of FIG. 2, and the trend update performed at 305 may include this additional data. In FIG. 3B for example, monitoring is further refined to distinguish false alarms from correct ones (e.g., in cases of exacerbation prediction) using the additional data to increase the confidence of or confirm a prediction or detection. This may be important in remote patient monitoring to intervene when it is adequately needed and not overload respiratory therapists or other clinicians with the need to check in when unnecessary. For example, elevated respiratory rate, coupled with increased cough, and lower SpO₂ is a better indicator of a potential AE-COPD event than increased respiratory rate alone. As a specific, non-limiting example, an embodiment may monitor sensor data from a plurality of sensors. In a first monitoring time window (A), sensor data may indicate an increased respiratory rate from increased physical activity, where the body achieves required SpO₂ and there is no need to generate an alert or indication, e.g., per coded rule. In a second monitoring time window (B), increased respiratory rate is again seen and may be explained or associated with increased physical activity, but the body does not receive the required SpO₂ and therefore a tailored alert or indication may be provided, e.g., alert for potential disease progression and associated recommendations, such as oxygen therapy or medications. In a third monitoring time window (C), increased respiratory rate is associated in time with sensor data indicating an increase in cough frequency and/or intensity, along with lowered SpO₂ but in the absence of increased physical activity. Therefore, an alert or indication is provided related to this combination of signals, e.g., an alert may be generated predicting an exacerbation and/or an indication of a detected exacerbation may be provided.

From the examples of FIG. 2A-D, it becomes evident that signal processing applied to the different sensor data (e.g., tidal breathing, cough, sneeze, etc.) may be used to distinguish between breathing events with granularity. For example, the sensor data may be separated through time and frequency waveform analysis followed by machine learning algorithms that rely on physiologically sound markers/features, such as characteristic waveform features, increased heart rate, etc.

In one example, peak and trough detection could be utilized to commence the detection of the different breathing events. A peak detection process could find a peaks within an interval of time, e.g., every 2 seconds (which is close to natural breathing). Following peak detection, a local search method can be applied to define an estimated start and end of those signals (tidal breathing, cough, sigh, sneezing, etc.) based on trough location around the determined peak. Thus, at this stage, each specific waveform is separated from the other and each signal/waveform can be analyzed separately or more specifically, physiological features of each waveform could be determined and output as descriptive metadata and used for analysis.

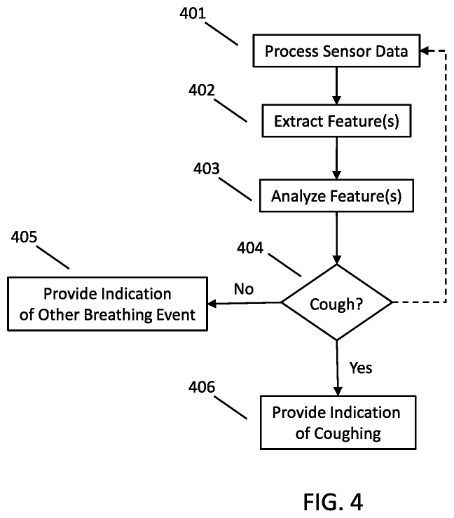

Referring to FIG. 4, an example process of using features extracted from sensor data is provided. After sensor data is obtained it may be processed at 401, for example segmentation of the signal applied to separate the inspiratory phase from the expiratory phase based on the peak (i.e., start of segment to peak will be the inspiration while peak to end of segment will be the expiration). This will allow for feature extraction at 402 and creation of associated descriptive metadata related to the features of the waveform. As another example, a high pass filter (with cutoff frequency around 25 breath/min) and/or a frequency spectrum is applied at 401 to the expiration phase. Normal breathing and a sigh will not have high frequency content above the threshold while the cough will; hence, allowing their differentiation and filtering, if desired.

Following any processing at 401, e.g., for filtering out signal features, segmenting the waveforms for further analysis, etc., feature extraction may be performed at 402. For example, a process performed at 402 may include calculating slopes of characteristic cough events. Referring to FIG. 2A, drawing an imaginary line from the peaks 210a (glottis closure peak) and 230a (expulsion peak) to the trough 220a on each side, provides the slope of each phase (glottis closure and intercostal/abdominal contraction, and glottis opening with explosive expulsion), which may be calculated along with a ratio of slopes and stored as descriptive metadata. As may be appreciated, these are examples of potentially useful features of many others. Features that may be characteristic of a breathing event include, but are not limited to, the ratio of slopes or the standard deviation between the slope and the segment itself, which can also be calculated as descriptive metadata. By way of specific example, a normal tidal volume will have similar slopes in the inspiration and expiration phase for all such segments (slope ratio of about 1.0), with only a small standard deviation (near 0). In contrast, a cough will exhibit a higher slope in the inspiration phase (e.g., larger than 1.5 times) and consequently a larger slope ratio (about 1.5). Further a cough will exhibit a larger standard deviation (SD) in the expiratory phase (e.g., SD larger by about 2.0).

Furthermore, the timing of or natural frequency and amplitude of the signals may be calculated at 402. These features could be used to analyze the features at 403, e.g., according to a rule, such as a rule coded to help differentiate between normal tidal breathing, sneezing, coughs, and sighs. For instance, normal tidal breathing should occur at physiological frequencies (e.g., around 10-20 breaths/min). However, as illustrated in FIG. 2D, normal tidal breaths will have a smaller amplitude and shorter time compared to a sigh. As illustrated in FIG. 2A, a cough reflex conversely will have a higher frequency compared to tidal breathing and may be of shorter duration, for example about 1.0 seconds or less, as compared to a sigh due to the large amount of lung volume rapidly expelled with each cough reflex. Each cough reflex in turn brings the individual closer to or past their functional residual capacity (FRC) point and potentially into their expiratory reserve volume (ERV) which would necessitate an inspiration effort prior to additional cough efforts, which is likely to aid in the classification effort.

The number of spikes or peaks in the expiratory phase may also calculated at 402 to perform analysis at 403. As observed in FIG. 2D, normal breathing has one spike 210d (a single peak at the start of the expiration phase) while, as illustrated in FIG. 2A, a single cough holds multiple close spikes 210a, 230a in the expiration phase. These features extracted at 402 could differentiate between the two at 403 and be used to decide at 404 as to whether a cough is present, which may be indicated at 406, or some other breathing event is observed in the sensor data, which may be indicated at 405. A cough could further be validated at 404 by searching for and validating the existence of a particular cough characteristic, such as three distinct peaks and their relative peak values and/or timing (e.g., the first peak is higher than the second peak).

Therefore, an embodiment uses identifiable segments that have a set of physiologically extracted features as the input to a decision module, which may be rules-based, a machine learning model, or a combination thereof, to classify each segment to its appropriate class (e.g., tidal breathing, cough, sneeze, etc.). In the example of a trained model, example features as described herein in connection with FIG. 2A-D, as well as others (dependent on the breathing event of interest) may be calculated on a set of data and utilized to train, test, and validate a machine learning model via different methods (e.g., linear regression, logistic regression, random forest, etc.). The model that achieves best performance can then be utilized as the classifier deployed, e.g., to a device such as device 102 of FIG. 1.

In an embodiment, a baseline of values for individual features and feature trends may be established as well as associated thresholds, which may be adjusted and utilized to differentiate unique segments or trends for each patient or patient type or sub-population. For example, baselines for features extracted at 402 may be established when a patient is stable and used to evaluate changes during feature analysis at 403. In one example, a baseline may be set on a personalized basis for each patient to determine baseline features of interest, such as slopes, respiratory rates, relative tidal volumes based on force peaks, etc. Thereafter, any deviation from a baseline may be used to determine breathing events at 404, e.g., indication of coughs, sighs, sneezes, and the like. Similarly, as illustrated in FIG. 3A, trend or monitoring data may be used to establish a baseline, where deviation from a recent time window of observations is utilized to indicate a predicted event or used for event detection. Such personalization may require more time and effort but in turn could insure higher performance. To offset this requirement for additional data collection, in an embodiment data for establishing a baseline could be collected during sleep, as in that case coughs are generally suppressed, and the patient is mainly stable. Furthermore, if some signals are labeled (e.g., seen by therapists and determined as cough, sneeze, etc.) then the thresholds can be further refined, e.g., reduced.

It may also be appreciated that an ability to accurately distinguish between breathing events may be used as a basis to refine additional measurements or metrics. By way of example, one or more identified coughs may be filtered out of a respiratory rate count. By way of specific example, a periodic or quasiperiodic waveform or other metadata associated with a peal of two or more coughs may be recognized as part of a single respiratory event (i.e., a single staggered exhalation) rather than erroneously classified as representing multiple breathing cycles, improving the accuracy of a respiratory rate determination.

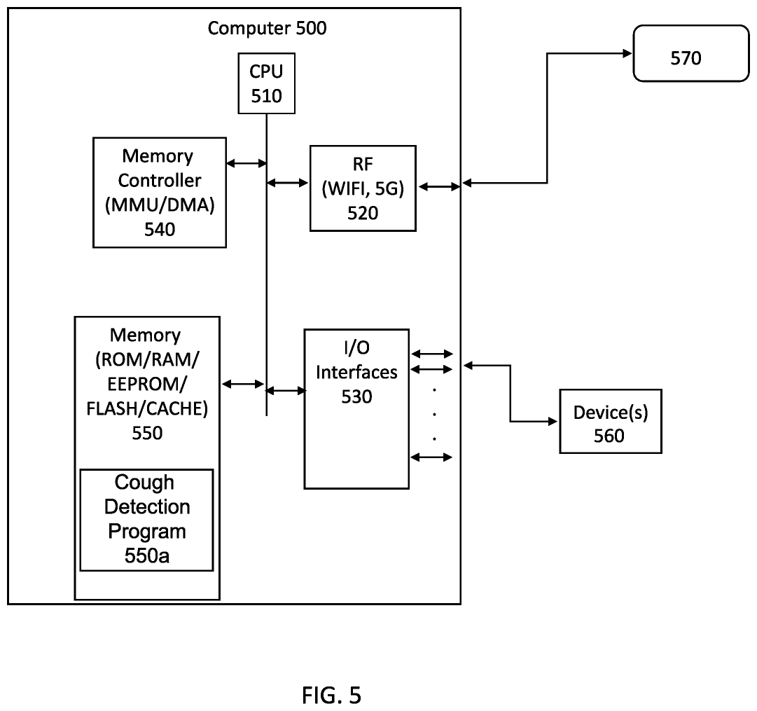

Referring to FIG. 5, it will be readily understood that certain embodiments can be implemented using any of a wide variety of devices or combinations of devices and components. In FIG. 5 an example of a computer 500 and its components are illustrated, which may be used in a device for implementing the functions or acts described herein, e.g., performing waveform analysis, combination or composite signal analysis for detection of cough or other breathing events. In addition, circuitry other than that illustrated in FIG. 5 may be utilized in one or more embodiments. The example of FIG. 5 includes certain functional blocks, as illustrated, which may be integrated onto a single semiconductor chip to meet specific application requirements.

One or more processing units are provided, which may include a central processing unit (CPU) 510, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 510 may comprise a single integrated circuit comprising several units, the design and arrangement of which vary according to the architecture chosen.

Computer 500 also includes a memory controller 540, e.g., comprising a direct memory access (DMA) controller to transfer data between memory 550 and hardware peripherals. Memory controller 540 includes a memory management unit (MMU) that functions to handle cache control, memory protection, and virtual memory. Computer 500 may include controllers for communication using various communication protocols (e.g., I²C, USB, etc.).

Memory 550 may include a variety of memory types, volatile and nonvolatile, e.g., read only memory (ROM), random access memory (RAM), electrically erasable programmable read only memory (EEPROM), Flash memory, and cache memory. Memory 550 may include embedded programs, code and downloaded software, e.g., a cough detection program 550a that provides coded methods such as illustrated in FIG. 3 and/or FIG. 4, which may include artificial neural network program(s) trained using FSC waveforms or descriptive metadata useful in producing predetermined classifications for breathing events as described herein. By way of example, and not limitation, memory 550 may also include an operating system, application programs, other program modules, code, and program data, which may be downloaded, updated, or modified via remote devices.

A system bus permits communication between various components of the computer 500. I/O interfaces 530 and radio frequency (RF) devices 520, e.g., WIFI and telecommunication radios, may be included to permit computer 500 to send and receive data to and from remote devices using wireless mechanisms, noting that data exchange interfaces for wired data exchange may be utilized. Computer 500 may operate in a networked or distributed environment using logical connections to one or more other remote computers or databases 570. The logical connections may include a network, such local area network (LAN) or a wide area network (WAN) but may also include other networks/buses. For example, computer 500 may communicate data with and between sensor device(s) 560 collecting sensor data as input for one or more artificial neural networks, training programs for training the same, etc. It will be appreciated by those having skill in the art that artificial neural networks such as those described herein, once trained, may be provided, and used on a local device, e.g., computer 500, which may take the form of an end user device such as a smartphone, tablet, desktop computer, etc.

Computer 500 may therefore execute program instructions or code configured to obtain, store, and analyze sensor data and perform other functionality of the embodiments, as described herein. A user can interface with (for example, enter commands and information) the computer 500 through input devices, which may be connected to I/O interfaces 530. A display or other type of device may be connected to the computer 500 via an interface selected from I/O interfaces 530.

It should be noted that the various functions described herein may be implemented using instructions or code stored on a memory, e.g., memory 550, that are transmitted to and executed by a processor, e.g., CPU 510. Computer 500 includes one or more storage devices that persistently store programs and other data. A storage device, as used herein, is a non-transitory computer readable storage medium. Some examples of a non-transitory storage device or computer readable storage medium include, but are not limited to, storage integral to computer 500, such as memory 550, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. The word “about” or similar relative term as applied to numbers includes ordinary (conventional) rounding of the number with a fixed base such as 5 or 10.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method, comprising:
obtaining, using a set of one or more processors, sensor data comprising one or more of a sensor signal received from a force sensor worn by a patient and descriptive metadata of the sensor signal;
distinguishing, using the set of one or more processors, between a cough and another breathing event of the patient based on the sensor data; and
providing, using the set of one or more processors, an indication of a cough.

2. The method of claim 1, wherein the another breathing event comprises one or more of a sneeze, throat clearing, a sigh, and tidal breathing.

3. The method of claim 1, wherein the distinguishing comprises utilizing one or more features of the sensor data to identify a cough characteristic associated with inspiration.

4. The method of claim 1, wherein the cough characteristic comprises a signal morphology that occurs after inspiration.

5. The method of claim 3, wherein the cough characteristic comprises one or more of:
a pair of signal peaks occurring within a predetermined time period; and
a ratio of slopes relating one of the pair of signal peaks prior to a trough and another of the pair of signal peaks following the trough.

6. The method of claim 5, wherein the predetermined time period is less than about 1.0 seconds.

7. The method of claim 5, wherein the ratio is about 1.5 or more.

8. The method of claim 5, wherein the cough characteristic comprises a standard deviation of slopes relating signal peaks to respective troughs.

9. The method of claim 5, wherein the cough characteristic comprises a predetermined pattern of signal peak intensities.

10. The method of claim 1, wherein the obtaining comprises obtaining the sensor data from a force sensing capacitor.

11. The method of claim 1, wherein:
the obtaining comprises obtaining sensor data from two or more sensors; and
the distinguishing comprises using signal data of the two or more sensors.

12. The method of claim 11, wherein the two or more sensors comprise one or more of: (a) a resistive, capacitive, inductive, or fiber-optic strain sensor; (b) an impedance sensor; (c) a heart rate sensor; and (d) one or more movement sensors comprising an accelerometer, a gyroscope, a magnetometer, or an inertial measurement unit (IMU).

13. The method of claim 1, wherein the distinguishing comprises:
identifying one or more features in training sensor data;
providing the training sensor data to a model based on the one or more features; and
using the model after training to classify the sensor data as a cough or another breathing event.

14. A system, comprising:
a wearable force sensor;
a set of one or more processors; and
a memory operatively coupled to the set of one or more processors and comprising code executable by the set of one or more processors, the code comprising:
code that obtains sensor data from the wearable force sensor comprising one or more of a sensor signal and descriptive metadata of the sensor signal;
code that distinguishes between a cough and another breathing event based on the sensor data; and
code that provides an indication of a cough.

15. A computer program product, comprising:
a non-transitory storage device operatively coupled to a processor and comprising code executable by the processor, the code comprising:
code that obtains sensor data from a wearable force sensor comprising one or more of a sensor signal and descriptive metadata of the sensor signal;
code that distinguishes between a cough and another breathing event based on the sensor data; and
code that provides an indication of a cough.
